(19) United States
(12) Patent Application Publication
(10) Pub. No.: US 2005/0059867 A1
(43) Pub. Date: Mar. 17, 2005

(54) Method for monitoring temperature of patient

(76) Inventor: Chung Yuan Cheng, Hsin Chuang City (TW)

Correspondence Address: Chung Yuan CHENG, P.O. Box 4-67, Hsin Chuang, Taipei, 242, TW

(21) Appl. No.: 10/662,652

(22) Filed: Sep. 13, 2003

(51) Int. Cl. A61B 5/00

(52) U.S. Cl. 600/300; 600/549; 374/120

(57) ABSTRACT

A method for remotely measuring body temperature of persons and for watching the persons, includes attaching one or more temperature measuring devices to one or more persons or patients, to detect body temperatures or locations or identifications of the persons or patients. The information detected by the temperature measuring devices may be sent to a monitoring device via a blue tooth receiver device, to receive and monitor and watch the changing of the body temperature or locations or identifications of the persons or patients wirelessly or remotely, without going to see and to contact with the persons or patients.

### BACKGROUND OF THE INVENTION

1. Field of the Invention

The present invention relates to a method for watching or monitoring body temperatures of users, and more particularly to a method for detecting or measuring and watching or monitoring the temperature or vibrations of people wirelessly or remotely with a blue tooth body temperature monitoring facility or system.

2. Description of the Prior Art

Normally, for taking care of or for watching or monitoring the patients in or out of hospitals, the nurses and/or the doctors have to go and see the patients and to detect or measure the body temperature of the patients many times everyday. The nurses and/or the doctors are thus required to take a lot of time to walk to the patients, and to detect or measure the body temperature of the patients.

Conventionally, the nurses and/or the doctors have to attach the typical thermometers onto the patients or to direct the typical infrared thermometers against the patients, in order to detect or measure the body temperatures of the users or of the patients, such that the nurses and/or the doctors have to contact with the patients directly.

However, for infectious diseases, such as the recently and widely separated severe acute respiratory syndrome (SARS), it will be dangerous for the nurses and/or the doctors to contact with the patients directly. It has been reported that many nurses and doctors have been infected during the SARS environment, due to directly contact with the patients.

During the SARS environment or situation, many many people have to be sensed or detected or measured before the people may go into the buildings, buses, airplanes, boats, or the like. However, it may take a lot of man power or labor to sense or detect or measure the body temperatures of the users.

The present invention has arisen to mitigate and/or obviate the afore-described disadvantages of the conventional thermometers.

### SUMMARY OF THE INVENTION

The primary objective of the present invention is to provide a method for detecting or measuring and watching or monitoring the temperature of people wirelessly or remotely with a blue tooth body temperature monitoring facility or system.

The other objective of the present invention is to provide a method for detecting or measuring and watching or monitoring the temperature of people wirelessly or remotely, to allow the nurses or doctors or parents to know and to watch the body temperatures of people wirelessly or remotely, and to service the patients or children right away.

In accordance with one aspect of the invention, there is provided a method for remotely measuring body temperature of persons and for watching the persons, the method comprising attaching at least one temperature measuring device to the person, to detect body temperature of the person, providing and arranging at least one blue tooth receiver device to receive the body temperature of the person detected by the temperature measuring devices, and providing a monitoring device to communicate with the temperature measuring device via a network system, to receive the body temperature of the person detected by the temperature measuring device, from the blue tooth receiver device, and to allow care nurses or doctors or parents or users to monitor or to watch the body temperature of the person wirelessly or remotely, without going to see and to contact with the person.

An identification and/or a location of the person may further be provided and sent to the monitoring device with the temperature measuring device, via the network system and the blue tooth receiver device, to allow the care nurses or doctors or parents or users to monitor or to watch the identification and/or the location of the person wirelessly or remotely, without going to see and to contact with the person.

The detected body temperature of the person may be transferred to a blue tooth signal for transmitting to the blue tooth receiver device, and for allowing the blue tooth signal from the temperature measuring device to be read by the blue tooth receiver device.

The blue tooth receiver device may search for the temperature measuring devices, to check whether the temperature measuring devices are located within the range of the blue tooth receiver device or not.

The temperature measuring device may compare the body temperature detected by from the blue tooth receiver device with a limit of endurable body temperature, to determine whether the detected body temperature exceeds the limit of endurable body temperature or not, and may display the detected body temperature when required, and may generate warning signals to warn the care persons when the detected body temperature exceeds the limit of endurable body temperature, and/or when the patients are moved out of the watching range.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a schematic view illustrating a method in accordance with the present invention, for detecting or measuring and watching or monitoring the temperature or vibrations of people wirelessly or remotely with a blue tooth body temperature monitoring facility or system;

FIG. 2 is a schematic view similar to FIG. 1, illustrating a simplified arrangement to conduct the method in accordance with the present invention to detect or measure or watch or monitor the temperature of people wirelessly or remotely with the blue tooth body temperature monitoring facility or system;

FIG. 3 is a block diagram illustrating the measuring or detecting processes with the temperature monitoring facility;

FIG. 4 is a block diagram illustrating the watching or monitoring processes with the temperature monitoring facility; and

FIG. 5 is a block diagram illustrating the receiving and displaying processes with the temperature monitoring facility.

### DETAILED DESCRIPTION OF THE PREFERRED EMBODIMENT

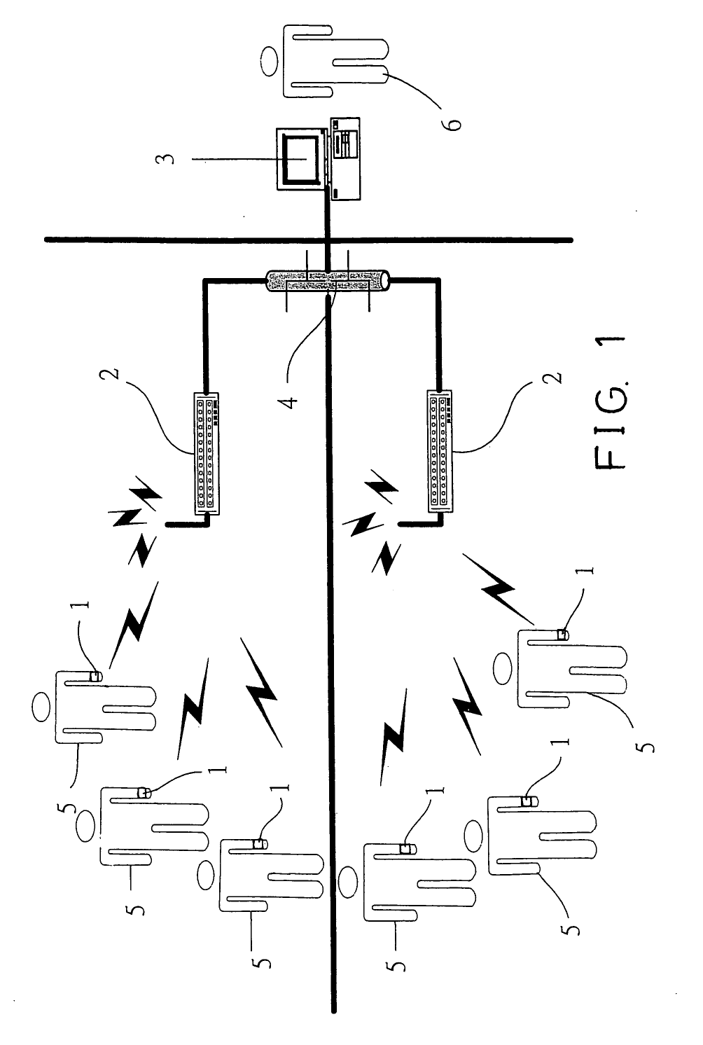

Referring to the drawings, and initially to FIG. 1, a method in accordance with the present invention is provided for detecting or measuring and watching or monitoring the temperature of people wirelessly or remotely with a blue tooth body temperature monitoring facility or system.

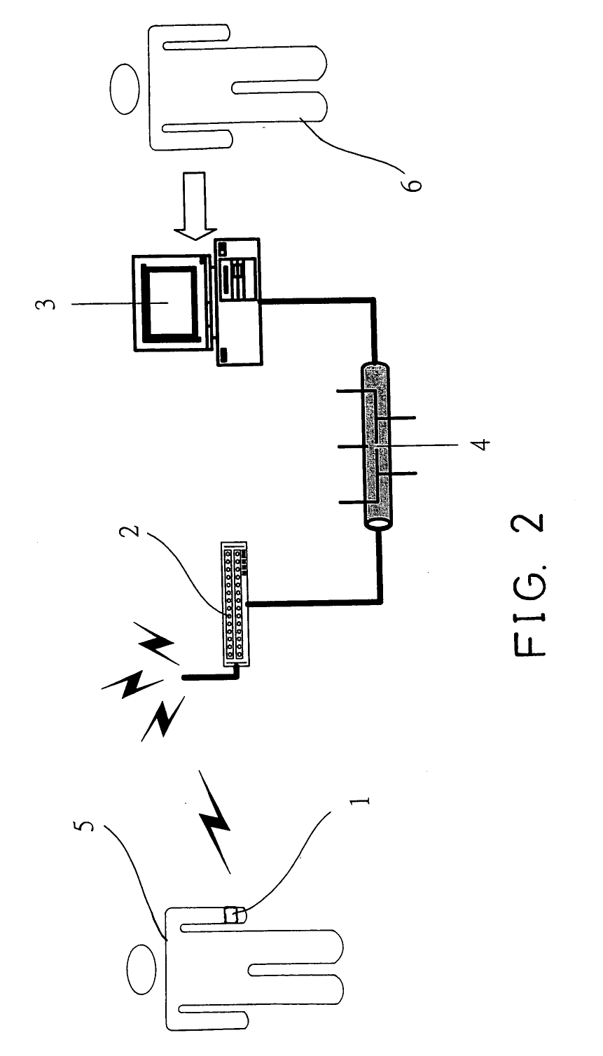

The blue tooth body temperature monitoring system includes one (FIG. 2) or more temperature measuring devices 1 (FIG. 1) for attaching onto various persons or users or children or old people or patients 5, such as for attaching to the waist portions of the patients or persons 5, to detect or measure the body temperature of the patients or persons 5, and/or to locate the patients or persons 5.

For example, the temperature measuring devices 1 may be used for providing or identifying the identification of the users or patients 5, the body temperature of the patients or persons 5, the time to detect and measure the body temperature of the patients or persons 5, etc.

The blue tooth body temperature monitoring system further includes one (FIG. 2) or more blue tooth receiver devices 2 (FIG. 1) disposed or arranged in various positions or locations, to receive the signals or information of the body temperature of the patients or persons 5 from the temperature measuring devices 1 wirelessly and remotely.

The blue tooth body temperature monitoring system further includes a watching or monitoring device 3 coupled to or communicating with the blue tooth receiver devices 2 via a network system 4, such as ethernet system 4 or the like, either via cables or wirelessly or remotely.

The parents or doctors or nurses 6 may thus use the watching or monitoring device 3 to know and to watch the body temperatures and/or the information of the patients 5 via the watching or monitoring device 3 wirelessly or remotely, and may then to take action and to service the patients or persons 5 right away.

It is to be noted that the blue tooth receiver devices 2 may be disposed or arranged in various positions or areas or locations, in order to receive the signals or information of the body temperature of the persons 5 that are located in different areas or locations, for allowing the body temperature and the other information of the persons 5 in various areas or locations to be transmitted to the watching or monitoring device 3 wirelessly and remotely via the blue tooth receiver devices 2 and the network system 4.

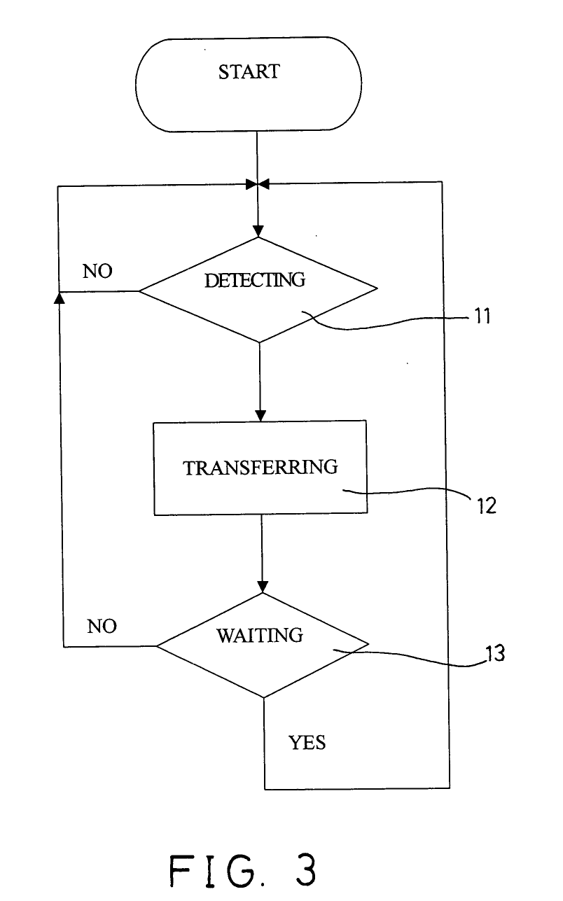

As shown in FIG. 3, in operation, the temperature measuring devices 1 may be used, in process 11, to detect or to measure the body temperature of the patients or persons 5, etc., and/or to provide or identify the identification or information of the users or patients 5, and/or the time to detect and measure the body temperature of the persons or patients 5.

The information or identification or the body temperature of the patients or persons 5, and/or the time to detect and measure the body temperature of the persons or patients 5 may then be collected and transferred or converted into such as the blue tooth format or signals, in process 12, and then wait for the information to be sent to the blue tooth receiver devices 2, in process 13.

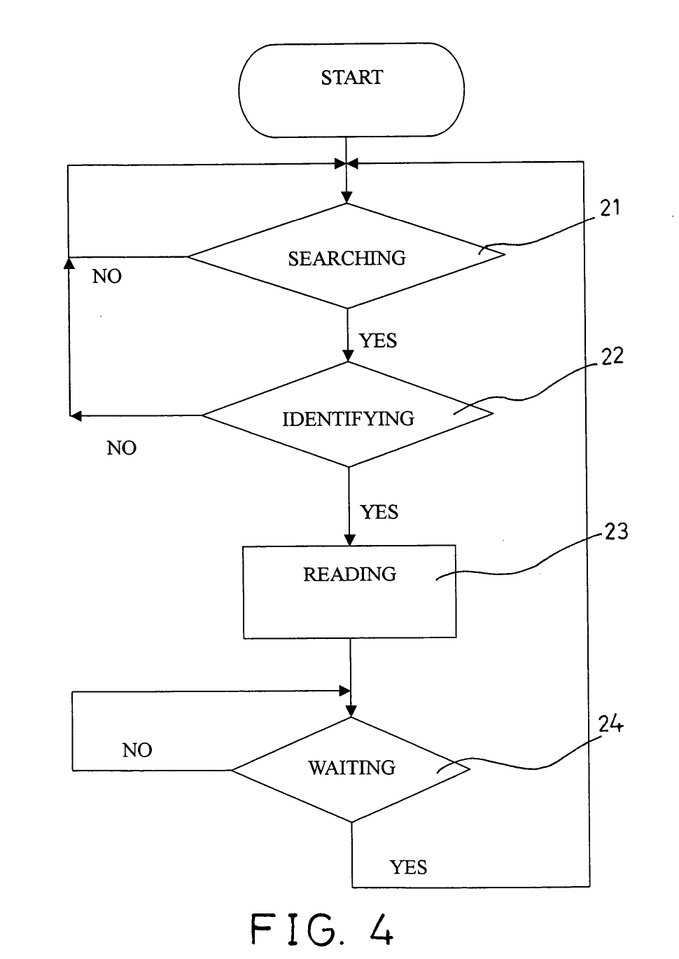

As shown in FIG. 4, illustrated is the information receiving and transmitting processes of the blue tooth receiver devices 2. For example, in process 21, the blue tooth receiver devices 2 may search for the temperature measuring devices 1, to check whether any temperature measuring devices 1 or patients or persons 5 are located within the area or range of the blue tooth receiver devices 2 respectively.

When the blue tooth receiver devices 2 receive the information or identification or the body temperature of the patients or persons 5, and/or the time to detect and measure the body temperature of the persons or patients 5, the blue tooth receiver devices 2 will first identify the number and/or the information or identification of the persons or patients 5, in process 22.

After the number and/or the information or identification of the persons or patients 5 have been identified in process 22, the blue tooth receiver devices 2 will then, in process 23, read the information or identification or the body temperature of the patients or persons 5, and/or the time to detect and measure the body temperature of the persons or patients 5.

The blue tooth receiver devices 2 may also be used to transfer or convert the blue tooth format or signals into such as the digital signals suitable for being sent to the watching or monitoring device 3 via the network system 4, and then wait for the information to be received or taken by the watching or monitoring device 3, in process 24.

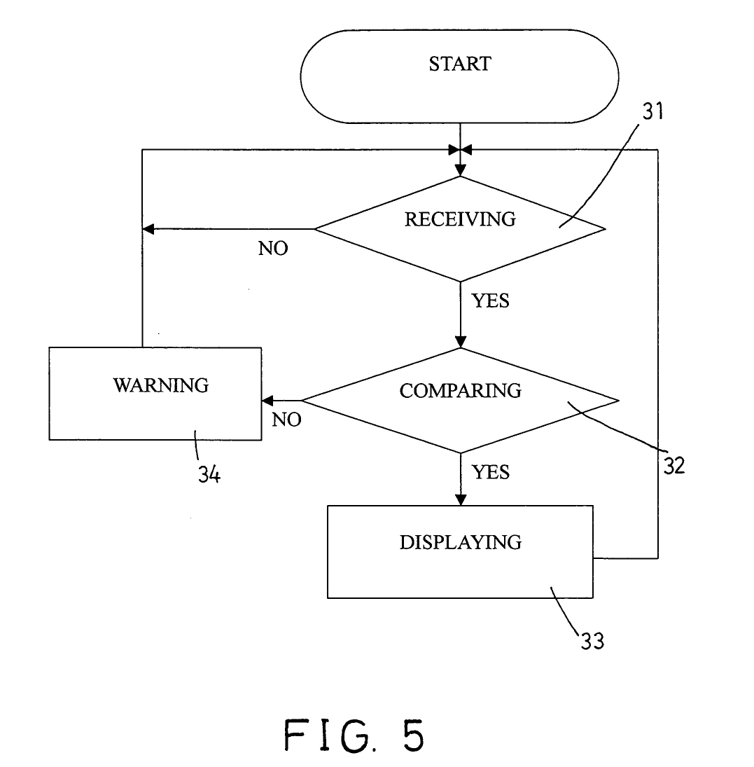

Referring next to FIG. 5, illustrated is the information receiving and processing and displaying processes by the watching or monitoring device 3. The watching or monitoring device 3 may first receive the information or identification or the body temperature of the patients or persons 5 from the blue tooth receiver devices 2, in process 31.

After receiving the information or identification or the body temperature of the patients or persons 5 from the blue tooth receiver devices 2, the watching or monitoring device 3 may then compare the newly detected body temperatures of the patients or persons 5 with the stored or previously detected informatoin, in process 32.

For example, the watching or monitoring device 3 may check whether the newly detected body temperatures are abnormal, or have been increased over the previous detected body temperatures or the predetermined or set limit of the endurable body temperature or not; and/or to detect whether the patients or persons 5 has left the predetermined areas or locations or positions or not.

If the newly detected body temperatures are normal or are within the limit or the predetermined or endurable body temperature, or if the patients or persons 5 are still located within the predetermined areas or locations or positions, the information or identification or the location or position or the body temperature and/or the changing of the body temperature of the patients or persons 5 may then be displayed in the watching or monitoring device 3, in process 33, for allowing the parents or doctors or nurses 6 to watch or to monitor the body temperature of the patients or persons 5 at any time.

On the contrary, when the newly detected body temperatures are abnormal or are out of the limit or the predetermined or endurable body temperature, or when the patients or persons 5 are moved out of the predetermined areas or locations or positions, a warning process 34 may be conduct to generate warning signals to the parents or doctors or nurses 6.

For example, the watching or monitoring device 3 may generate warning signals by such as speaker, buzzers, indicating lights, vibrating devices, etc., in order to warn the parents or doctors or nurses 6 that the body temperatures of the patients or persons 5 are abnormal or are out of the limit or the predetermined or endurable body temperature, or that the patients or persons 5 are moved out of the predetermined areas or locations or position, for example.

The nurses and/or the doctors and/or the parents 5 may thus watch or monitor or detect or know the body temperatures of the patients or persons 6 at any time wirelessly or remotely, without going to see and to contact with the patients or persons 5. In addition, the nurses or doctors or parents may take actions to service the patients or children or old people at the very first time when required.

Accordingly, the blue tooth body temperature monitoring system may be used for watching or monitoring the body temperature of people wirelessly or remotely, and thus for allowing the nurses or doctors or parents or the other care people to take action or to make service to the patients or children right away.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

1. A method for remotely measuring body temperature of persons and for watching the persons, said method comprising:
attaching at least one temperature measuring device to the person, to detect body temperature of the person,
providing and arranging at least one blue tooth receiver device to receive the body temperature of the person detected by said at least one temperature measuring devices, and
providing a monitoring device to communicate with said at least one temperature measuring device via a network system, to receive the body temperature of the person detected by said at least one temperature measuring device, from said at least one blue tooth receiver device.

2. The method as claimed in claim 1 further comprising providing and sending an identification of the person to said monitoring device with said at least one temperature measuring device, via said network system and said at least one blue tooth receiver device.

3. The method as claimed in claim 1 further comprising providing and sending a location information of the person to said monitoring device with said at least one temperature measuring device, via said network system and said at least one blue tooth receiver device.

4. The method as claimed in claim 1 further comprising transferring the detected body temperature of the person to a blue tooth signal for transmitting to said at least one blue tooth receiver device.

5. The method as claimed in claim 4 further comprising reading said blue tooth signal from said at least one temperature measuring device with said at least one blue tooth receiver device.

6. The method as claimed in claim 1 further comprising searching for said at least one temperature measuring devices with said at least one blue tooth receiver device, to check whether said at least one temperature measuring device is located within a range of said at least one blue tooth receiver device.

7. The method as claimed in claim 1 further comprising comparing the body temperature detected by said at least one temperature measuring device from said at least one blue tooth receiver device with a limit of endurable body temperature, to determine whether the detected body temperature exceeds the limit of endurable body temperature or not.

8. The method as claimed in claim 7 further comprising displaying the detected body temperature.

9. The method as claimed in claim 7 further comprising generating a warning signal when the detected body temperature exceeds the limit of endurable body temperature.
